(19) United States
(12) Patent Application Publication
(10) Pub. No.: US 2021/0298886 A1
(43) Pub. Date: Sep. 30, 2021

(54) Animal Leg Support Brace Apparatus

(71) Applicant: Gary Creel, Kittanning, PA (US)

(72) Inventor: Gary Creel, Kittanning, PA (US)

(21) Appl. No.: 16/835,483

(22) Filed: Mar. 31, 2020

(51) Int. Cl. A61D 9/00; A61F 5/37

(52) U.S. Cl. CPC A61D 9/00; A61F 5/3715

(57) ABSTRACT

An animal leg support brace apparatus for facilitating animal leg injury recovery includes a wrap body. A pair of shoulder straps is coupled to the wrap body. Each shoulder strap has a strap proximal end coupled to the wrap body and a strap distal end. A first wrap engagement member and a second wrap engagement member are coupled to the wrap body and the pair of shoulder straps, respectively, to secure the wrap body around an animal's injured leg. A pair of strap engagement members is coupled to the pair of shoulder straps to secure the pair of shoulder straps around the animal's shoulders.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to animal medical braces and more particularly pertains to a new animal medical brace for facilitating animal leg injury recovery.

### (2) Description of Related Art Including Information Disclosed Under 37 CFR 1.97 and 1.98

The prior art relates to animal medical braces. Existing devices offer support for leg joints, such as hips and knees, particularly for the animals' back legs. The existing braces include support rods and pivot points for intentional limitation of movement, and often have an extensive strap system spanning across the animal's body. What is needed, and what the present invention provides, is a flexible wrap designed to prevent the animal from licking and irritating an injury by providing coverage, as well as a strap to hold the wrap in place and take weight off the injured leg.

### BRIEF SUMMARY OF THE INVENTION

An embodiment of the disclosure meets the needs presented above by generally comprising a wrap body having a wrap inner face, a wrap outer face, a wrap top edge, a wrap bottom edge, a wrap front edge, and a wrap back edge. A first wrap engagement member is coupled to the wrap inner face adjacent the wrap front edge. A pair of shoulder straps is coupled to the wrap body. Each shoulder strap has a strap proximal end coupled to the wrap outer face, a strap distal end, a strap inner side, and a strap outer side.

A second wrap engagement member is coupled to the strap proximal end of the pair of shoulder straps. The second wrap engagement member is selectively engageable with the first wrap engagement member. The first wrap engagement member and the second wrap engagement member are configured to secure the wrap body around an animal's injured leg. A pair of strap engagement members is coupled to the pair of shoulder straps. The pair of strap engagement members is coupled to the strap outer side of an inner strap of the pair of shoulder straps and to the strap inner side of an outer strap of the pair of shoulder straps. The pair of strap engagement members is selectively engageable and configured to secure the pair of shoulder straps around the animal's shoulders.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric view of an animal leg support brace apparatus according to an embodiment of the disclosure.

FIG. 2 is an isometric view of an embodiment of the disclosure.

FIG. 3 is a front elevation view of an embodiment of the disclosure.

FIG. 4 is an in-use view of an embodiment of the disclosure.

FIG. 5 is an in-use view of an embodiment of the disclosure.

### DETAILED DESCRIPTION OF THE INVENTION

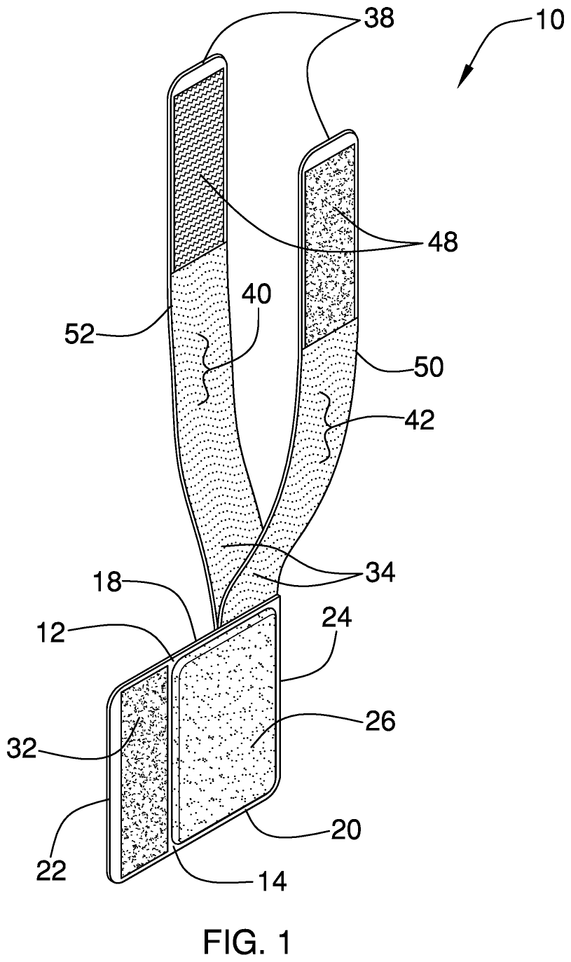

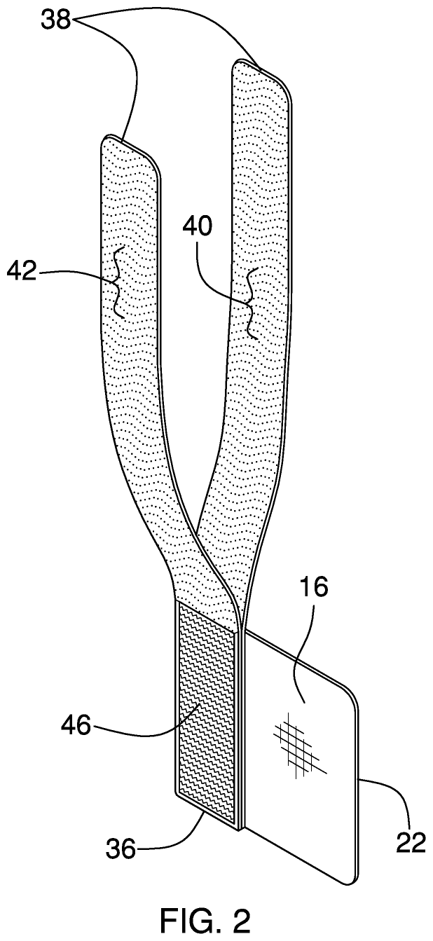

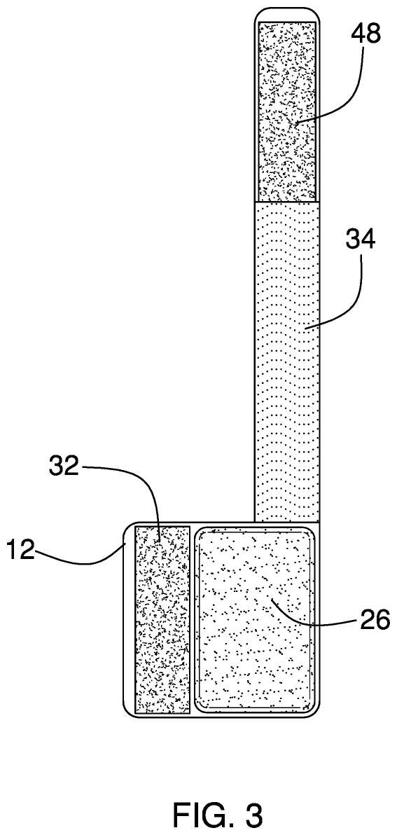

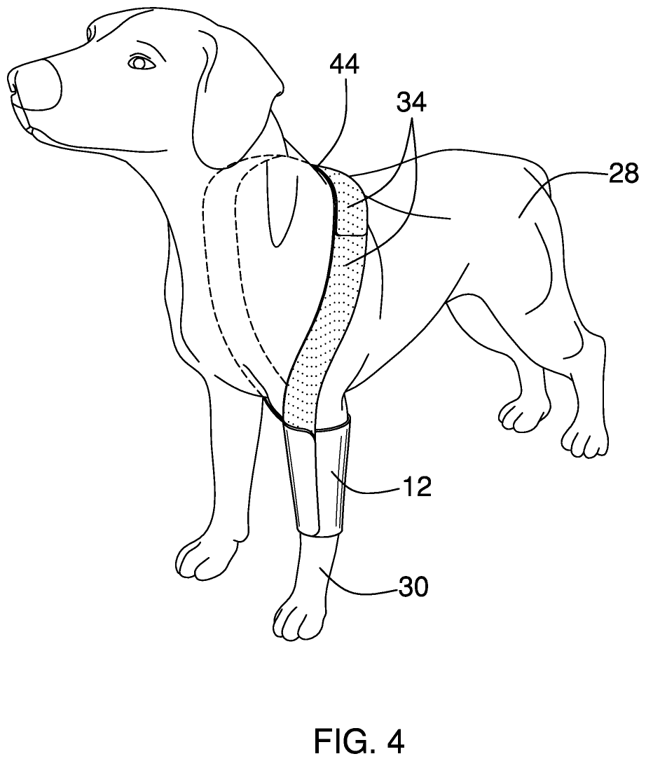

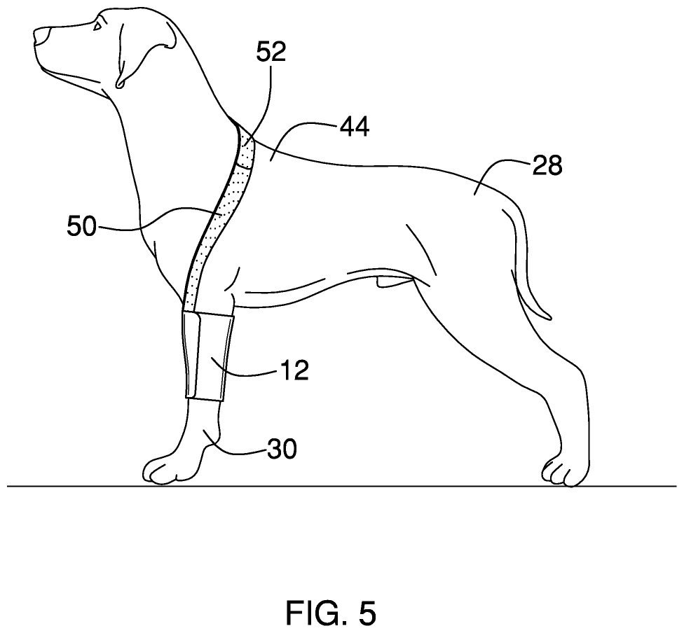

With reference now to the drawings, and in particular to FIGS. 1 through 5 thereof, a new animal medical brace embodying the principles and concepts of an embodiment of the disclosure and generally designated by the reference numeral 10 will be described.

As best illustrated in FIGS. 1 through 5, the animal leg support brace apparatus 10 generally comprises a wrap body 12 having a wrap inner face 14, a wrap outer face 16, a wrap top edge 18, a wrap bottom edge 20, a wrap front edge 22, and a wrap back edge 24. The wrap inner face 14 may have a padded portion 26 adjacent the wrap back edge 24. The wrap body 12 may have rounded corners for improved comfort. The wrap body 12 is sufficiently flexible to wrap tightly and is configured to allow an animal 28 to move. The padded portion 26 is dimensioned to fully encompass an injured leg 30 of the animal when wrapped.

A first wrap engagement member 32 is coupled to the wrap inner face 14 adjacent the wrap front edge 22. The padded portion 26 and the first wrap engagement member 32 may occupy over 90% of the wrap inner face 14.

A pair of shoulder straps 34 is coupled to the wrap body 12. Each shoulder strap 34 has a strap proximal end 36 coupled to the wrap outer face 16, a strap distal end 38, a strap inner side 40, and a strap outer side 42. The strap distal end 38 of each shoulder strap may have rounded corners for improved comfort. Each of the pair of shoulder straps 34 is sufficiently elastic to allow the animal 28 to move freely and to transfer weight from the animal's injured leg 30 to a shoulders 44 of the animal.

A second wrap engagement member 46 is coupled to the strap proximal end 36 of the pair of shoulder straps. The second wrap engagement member 46 is selectively engageable with the first wrap engagement member 32. The first wrap engagement member 32 and the second wrap engagement member 46 are configured to secure the wrap body 12 around the animal's injured leg 30. The wrap body 12 may thus cover an injured, stitched, or bandaged area to prevent the animal 28 from licking it.

A pair of strap engagement members 48 is coupled to the pair of shoulder straps 34. The pair of strap engagement members 48 is coupled to the strap outer side 42 of an inner strap 50 of the pair of shoulder straps and to the strap inner side 40 of an outer strap 52 of the pair of shoulder straps. The strap engagement members 48 may be coupled adjacent the strap distal end 38 of each shoulder strap and are selectively engageable. The pair of strap engagement members 48 is configured to secure the pair of shoulder straps 34 around the animal's shoulders 44.

Each of the strap engagement members 48, the first wrap engagement member 32, and the second wrap engagement member 46 may be hook-and-loop fasteners or another selectively engageable fastener such as, but not limited to, snaps, buttons, buckles, and the like.

In use, the wrap body 12 is wrapped around the animal's injured leg 30 with the padded portion 26 covering the injured area and the first wrap engagement member 32 engaging the second wrap engagement member 46. The outer strap 52 is then passed between the animal's front legs and engaged with the inner strap 50 on the animal's shoulders 44 using the pair of strap engagement members 48. The pair of shoulder straps 34 thus secure the wrap body 12 in place and also remove weight from the injured leg 30 to increase recovery time.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An animal leg support brace apparatus comprising:
a wrap body having a wrap inner face, a wrap outer face, a wrap top edge, a wrap bottom edge, a wrap front edge, and a wrap back edge;
a first wrap engagement member coupled to the wrap inner face adjacent the wrap front edge;
a pair of shoulder straps coupled to the wrap body, each shoulder strap having a strap proximal end coupled to the wrap outer face, a strap distal end, a strap inner side, and a strap outer side;
a second wrap engagement member coupled to the strap proximal end of the pair of shoulder straps, the second wrap engagement member being selectively engageable with the first wrap engagement member, the first wrap engagement member and the second wrap engagement member being configured to secure the wrap body around an animal's injured leg; and
a pair of strap engagement members coupled to the pair of shoulder straps, the pair of strap engagement members being coupled to the strap outer side of an inner strap of the pair of shoulder straps and to the strap inner side of an outer strap of the pair of shoulder straps, the pair of strap engagement members being selectively engageable, the pair of strap engagement members being configured to secure the pair of shoulder straps around the animal's shoulders.

2. The animal leg support brace apparatus of claim 1 further comprising the wrap inner face having a padded portion adjacent the wrap back edge.

3. The animal leg support brace apparatus of claim 2 further the padded portion and the first wrap engagement member occupying over 90% of the wrap inner face.

4. The animal leg support brace apparatus of claim 1 further comprising the wrap body being sufficiently flexible to wrap tightly and allow the animal to move.

5. The animal leg support brace apparatus of claim 1 further comprising each of the pair of shoulder straps being sufficiently elastic to allow the animal to move freely and to transfer weight from the animal's injured leg to the animal's shoulders.

6. The animal leg support brace apparatus of claim 1 further comprising the wrap body having rounded corners.

7. The animal leg support brace apparatus of claim 1 further comprising the strap engagement members being coupled adjacent the strap distal end of each shoulder strap.

8. The animal leg support brace apparatus of claim 1 further comprising the strap distal end of each shoulder strap having rounded corners.

9. An animal leg support brace apparatus comprising:
a wrap body having a wrap inner face, a wrap outer face, a wrap top edge, a wrap bottom edge, a wrap front edge, and a wrap back edge, the wrap inner face having a padded portion adjacent the wrap back edge, the wrap body having rounded corners, the wrap body being sufficiently flexible to wrap tightly and configured to allow an animal to move;
a first wrap engagement member coupled to the wrap inner face adjacent the wrap front edge, the padded portion and the first wrap engagement member occupying over 90% of the wrap inner face;
a pair of shoulder straps coupled to the wrap body, each shoulder strap having a strap proximal end coupled to the wrap outer face, a strap distal end, a strap inner side, and a strap outer side, the strap distal end of each shoulder strap having rounded corners, each of the pair of shoulder straps being sufficiently elastic to allow the animal to move freely and to transfer weight from the animal's injured leg to the animal's shoulders;
a second wrap engagement member coupled to the strap proximal end of the pair of shoulder straps, the second wrap engagement member being selectively engageable with the first wrap engagement member, the first wrap engagement member and the second wrap engagement member being configured to secure the wrap body around the animal's injured leg; and
a pair of strap engagement members coupled to the pair of shoulder straps, the pair of strap engagement members being coupled to the strap outer side of an inner strap of the pair of shoulder straps and to the strap inner side of an outer strap of the pair of shoulder straps, the strap engagement members being coupled adjacent the strap distal end of each shoulder strap, the pair of strap engagement members being selectively engageable, the pair of strap engagement members being configured to secure the pair of shoulder straps around the animal's shoulders.
